(19) United States
(12) Patent Application Publication
(10) Pub. No.: US 2015/0057941 A1
(43) Pub. Date: Feb. 26, 2015

(54) METHOD FOR COMPUTING A PARAMETER REPRESENTATIVE OF THE ENDURANCE OF A TENNIS PLAYER

(71) Applicants: Pierre MACE, LYON (FR); Fabien GAUTHIER, LYON (FR)

(72) Inventors: Pierre MACE, LYON (FR); Fabien GAUTHIER, LYON (FR)

(73) Assignee: BABOLAT VS, Lyon (FR)

(21) Appl. No.: 14/460,873

(22) Filed: Aug. 15, 2014

(30) Foreign Application Priority Data
Aug. 20, 2013 (FR) 1358082

(51) Int. Cl. A61B 5/00; G01P 3/00; A61B 5/11; G01P 15/00

(52) U.S. Cl. CPC A61B 5/4866; G01P 15/00; G01P 3/00; A61B 5/1118; A61B 5/7278; A61B 5/6895
USPC 702/19

(57) ABSTRACT

Method for calculating a parameter representing a tennis player's endurance during a game period including a series of game sessions, includes: a) measuring the linear acceleration and angular speed of the racket during each stroke hit by the player, b) calculating the power developed by the player during each stroke using linear accelerations and angular speeds measured in step a); c) calculating the player's average power defined as the average of power levels developed during each stroke calculated in step b); d) calculating the energy expended by the player during the last game session, the energy expended being equal to the number of strokes executed during the session multiplied by the average power calculated in step c) raised to a predetermined power, e) calculating the parameter, defined by the product of the number of game sessions per week during the game period, multiplied by the energy calculated in step d).

### FIELD OF THE INVENTION

The invention relates to a method for calculating a parameter representing the endurance of a tennis player during a game session.

### BACKGROUND OF THE INVENTION

In the practice of tennis, it is advantageous to be able to observe the performance, in particular physical, of a player. It is in particular interesting to know the change in the endurance according to the game intensity and frequency, in order to allow the player to improve his physical capacities.

### SUMMARY OF THE INVENTION

To this effect, the invention relates to a method for calculating a parameter representing the endurance of a tennis player during a game period comprising a series of game sessions. This method is characterised in that it comprises at least the steps consisting in:

a) measuring, using a system for measuring comprising sensors installed on a racquet used by the player, the linear acceleration and the angular speed of the racket during each stroke hit by the player during a game session;

b) calculating by means of the calculation unit, the power developed by the player during each stroke using linear accelerations and angular speeds measured in the step a);

c) calculating by means of the calculation unit, the average power developed by the player during the game session, with the average power being defined as the average of the power levels developed during each stroke by the player calculated in the step b);

d) calculating by means of the calculation unit, the energy expended by the player during the last game session, the energy expended being equal to the number of strokes executed by the player during the game session multiplied by the average power calculated in the step c) raised to a predetermined power (i);

e) calculating by means of the calculation unit, the value of the parameter, with this value being defined by the product of the number of game sessions carried out per week during the game period, multiplied by the energy calculated in the step d).

Thanks to the invention, a player can evaluate his capacity to link together game sessions with a high hitting intensity in a short interval of time, which allow him in particular to improve his endurance, for example during preparation before a competition.

According to advantageous but not mandatory aspects of the invention, such a method can incorporate one or several of the following characteristics, taken in any technically permissible combination:
The power to which the average power is raised in the step d) is chosen in such a way that it increases the weight of the average power in the calculation of the energy, and is more preferably between 0.1 and 4.
The number of sessions per week is calculated over a sliding game period comprising a predetermined number of game sessions.
The number of game sessions taken into account per week is limited by a maximum value.
The method comprises an additional step f) consisting in refining and in bringing the value of the parameter calculated in the step e) onto a predetermined scale and according to the formula:

$$Px = Px max \times {( \frac{P}{P max} )}^{j}$$

where Px is the new value of the parameter on the predetermined scale, Pxmax is the maximum value of the predetermined scale, Pmax is the maximum value of the parameter calculated in the step e), and j is a predetermined power.
In the step f), x is set to 100 and the parameter is calculated in the form of a percentage.
In the step f), the value of the predetermined power is between 0.1 and 1.
The value of the parameter is limited to a maximum value.
The game period over which the number of game sessions per week is calculated comprises a minimum number of game sessions.
The method comprises an additional step consisting in calculating the average of the values of the parameter over all of the game sessions taken into account of the game period.
The method comprises an additional step consisting in exporting, to a calculation interface in a device for calculating and displaying data, the data measured in the step a).
The method comprises a step consisting in limitating the increasing of the number of game sessions per week between two consecutive game sessions to a maximal value.

### BRIEF DESCRIPTION OF THE DRAWINGS

The invention shall be better understood and other advantages of the latter shall appear more clearly when reading the following description, of an embodiment of a method for calculating a parameter in accordance with its principle, and made in reference to the annexed drawings wherein:

FIG. 1 is a front view of a tennis racket making it possible to implement the method of calculation according to the invention; and

FIG. 2 is a block diagram of the method according to the invention.

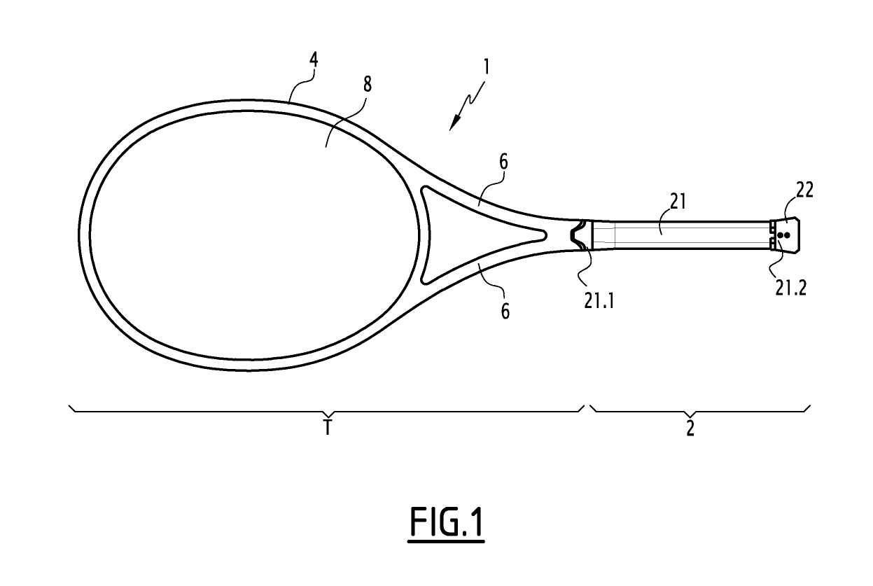

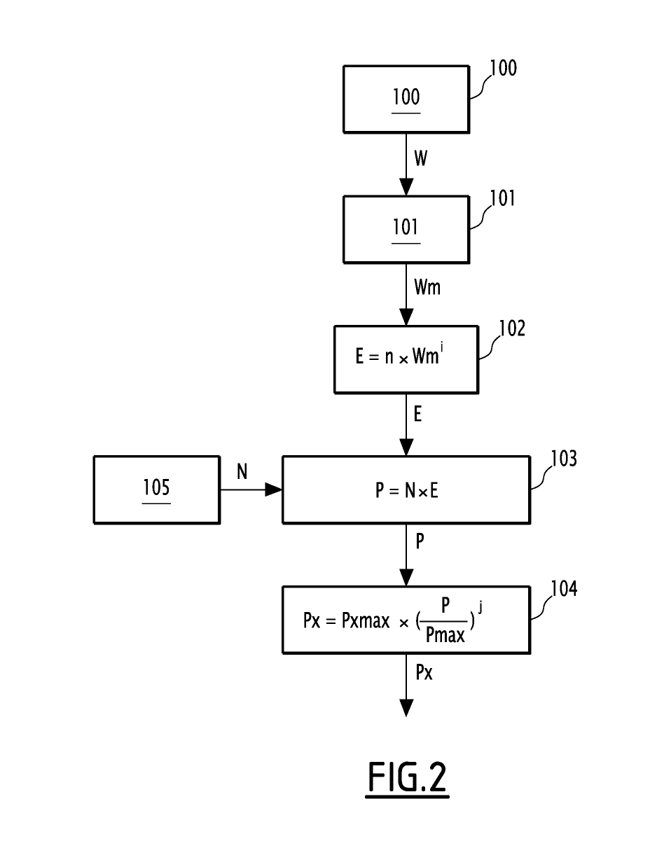

FIG. 1 shows a tennis racket 1, which conventionally comprises a grip 2 connected to a head T including a frame 4 inside of which is tensioned a stringed zone 8, formed of various transverse and longitudinal strings. The head T further comprises two connecting branches 6 that extend in a diverging manner from the grip 2 to the frame 4.

The grip 2 comprises a generally tubular body 21, made for example of carbon. Conventionally, the body 21 is covered by a thickness of foam surrounded by a grip, in order to improve the grasping of the grip. The body 21 of the grip 2 comprises a proximal longitudinal end 21.1, located on the side of the stringed zone 8 and connected to the connecting branches 6, and a distal longitudinal end 21.2, free and opposite the end 21.1. The end 21.2 is covered by a heel 22, generally designated by the term “butt cap”, which allows the hand of the player to not slide outside of the grip 2.

The racquet 1 is provided with a system for measuring game data, not shown, comprising measurement sensors able to measure, during the hitting of a ball with the racquet 1, the linear acceleration and the angular speed of the racket 1, according to three directions perpendicular to each other, fixed in relation to a terrestrial reference. For example, the system for measuring can include a triple-axis accelerometer and a triple-axis gyrometer

The rest of the description relates to a method for calculating a parameter representing the endurance of a tennis player, by means of the racquet 1.

During a game period that comprises several game sessions, game data is measured by the system for measuring, when a player carries out a series of strokes (forehand topspin stroke, serve, backhand slice, etc.). The data collected is more preferably exported to a calculation unit in a device for calculating and displaying data, for example a computer or a smartphone. For example, the calculation unit can be a microprocessor.

In a first step 100, the power W developed by the player for each stroke hit is calculated by the calculating unit. The power W is defined here as a specific quantity calculated using the ratio between the linear acceleration and the angular speed measured by the sensors of the system for measuring of the racquet 1. The power W is expressed preferably by a percentage, using a value between 0 and 1.

In a second step 101, the average power Wm developed by the player during the last game session is calculated by the calculation unit. The average power is defined as the average of the power levels W calculated in the step 100.

In a step 102, the energy E expended by the player during the last game session is calculated by the calculation unit. The energy E is equal to the number of strokes n executed by the player during the game session, multiplied by the average power Wm calculated in the step 101 raised to a power i, according to the relation E=n×Wmⁱ, where i is a predetermined power.

In a preferred embodiment, the power i is chosen in such a way that it increases the weight of the average power Wm in the calculation of the energy E. The power i is, more preferably, between 0.1 and 4. In the example described, as the average power Wm is a value less than 1, the power i can be chosen between 0.1 and 1 in order to increase the weight of the average power Wm. By way of example, i can be equal to 0.75. The value of the energy E can be multiplied by a correction coefficient in order to bring the value of energy E onto a predetermined scale.

In a step 103, the value of the parameter P is calculated by the calculation unit. The value P is defined by the product of the number N of game sessions carried out per week, during the game period, multiplied by the energy calculated in the step 102.

The parameter P is thus calculated for each game session, taking into account the data of the last game session and the playing frequency over a game period comprising the last game session.

The number N of game sessions per week is calculated over a sliding game period comprising a predetermined number of game sessions, for example six game sessions. This means that each time that the data concerning a new game session is recorded, the data concerning this new session replaces the oldest data of the six sessions considered.

The number N of sessions per week is calculated, in a step 105, by the calculation unit, by determining the number of weeks required to obtain six game sessions, then by establishing the number of corresponding game sessions per week.

If the total number of game sessions carried out is insufficient, the number N of sessions per week is calculated over a lower number of game sessions but at least equal to a minimum value, for example two game sessions.

In order to prevent the endurance parameter from reaching an incoherent value if the player multiplies the game sessions, the number N of game sessions per week is limited to a maximum value, for example seven game sessions per week.

Advantageously, the value of the parameter P is brought, in a step 104, onto a predetermined scale, to a value Px, for example a percentage P100. The new value Px is nevertheless refined in order to not penalise the players who play only about once a week, by means of the following formula:

$$Px = Px max \times {( \frac{P}{P max} )}^{j}$$

Where:
Px is the value of the parameter on a scale of 0 to a value x,
Pxmax is the maximum value of the scale chosen and
Pmax is the maximum value of the parameter calculated in the step 103.
j is a predetermined power.

The value of the power j is preferably between 0.1 and 1, which makes it possible to obtain an endurance value which does not excessively penalise the players who play a reduced number N of sessions per week, by defining a rapidly increasing hyperboloid curve for the low values of N.

In the example described, in order to obtain the percentage P100, x is set to 100 and Pxmax=100. In this case, the parameter P is written in the form of a percentage P100 given by the formula:

$$P 100 = 100 \times {( \frac{P}{P max} )}^{j}$$

Alternatively, the value of P can also be calculated on a scale from 0 to 50, by setting x=50 and by calculating P50, or a scale of 0 to 25, by setting x=25 and by calculating P25.

The maximum value Pmax is set, for example, to 10,000, in order to prevent incoherent results.

The endurance parameter P reflects the capacity of the player to practice game sessions with a high energy expenditure, by putting substantial power into his hits, and by repeating such sessions on a regular basis.

In an optional additional step, the average of the values of the parameter P over the six game sessions considered for the calculation of the parameter P can be calculated.

In order to prevent the value of the parameter P from increasing in a too brutal way if a player, which regularly plays, starts again to play regularly after a long inactivity period, the method comprises a step consisting in limiting the increasing of the number N of game sessions per week between two consecutive game sessions to a maximal value, for example two.

Thus, a player who played regularly and who had, for example, a number N equal to 7 and who, for any reason, has an inactivity period during which he won't play for two months, will see his number N and his parameter P fall. When he will start again his playing rhythm of prior his inactivity period, the calculation of the number N will still take into account the last game session of this player before his inactivity period, the number N remaining low until he plays six game sessions after his starting again. At this moment, the sliding game period of six game sessions on which the number N is calculated will no longer take into account the two months gap between the last game session before the inactivity period and the first game session following the inactivity period. The limitation of the increasing of the number N between two consecutive game sessions avoids that the number N increases brutally to 7, and that the parameter P increases too brutally. The number N will progressively increase to set back to its value of before the two months interruption.

1. Method for calculating a parameter representing the endurance of a tennis player during a game period comprising a series of game sessions, wherein it comprises at least steps consisting in:
a) measuring, using a system for measuring comprising sensors installed on a racquet used by the player, the linear acceleration and the angular speed of the racket during each stroke hit by the player during a game session;
b) calculating by means of the calculation unit, the power developed by the player during each stroke using linear accelerations and angular speeds measured in the step a);
c) calculating by means of the calculation unit, the average power developed by the player during the game session, with the average power being defined as the average of the power levels developed during each stroke by the player calculated in the step b);
d) calculating by means of the calculation unit, the energy expended by the player during the last game session, the energy expended being equal to the number of strokes executed by the player during the game session multiplied by the average power calculated in the step c) raised to a predetermined power;
e) calculating by means of the calculation unit, the value of the parameter, with this value being defined by the product of the number of game sessions carried out per week during the game period, multiplied by the energy calculated in the step d).

2. Method according to claim 1, wherein the power to which the average power is raised in the step d) is chosen in such a way that it increases the weight of the average power in the calculation of the energy and is more preferably between 0.1 and 4.

3. Method according to claim 1, wherein the number of sessions per week is calculated over a sliding game period comprising a predetermined number of game sessions.

4. Method according to claim 1, wherein the number of game sessions taken into account per week is limited by a maximum value.

5. Method according to claim 1, wherein it comprises an additional step f) consisting in refining and in bringing the value of the parameter calculated in the step e) onto a predetermined scale and according to the formula:
$$Px = Px max \times {( \frac{P}{P max} )}^{j}$$
Where:
Px is the new value of the parameter on the predetermined scale,
Pxmax is the maximum value of the predetermined scale
Pmax is the maximum value of the parameter calculated in the step e), and
j is a predetermined power.

6. Method according to claim 5, wherein in the step f), x is set to 100 and in that the parameter (P) is calculated in the form of a percentage (P100).

7. Method according to claim 5, wherein in the step f), the value of the predetermined power is between 0.1 and 1.

8. Method according to claim 1, wherein the value of the parameter is limited to a maximum value.

9. Method according to claim 1, wherein the game period on which the number of game sessions per week is calculated comprises a minimum number of game sessions.

10. Method according to claim 1, wherein it comprises an additional step consisting in calculating the average of the values of the parameter over all of the game sessions taken into account of the game period.

11. Method according to claim 1, wherein it comprises an additional step consisting in exporting, to a calculation interface in a device for calculating and displaying data, the data measured in the step a).

12. Method according to claim 1, wherein it comprises a step consisting in limitating the increasing of the number (N) of game sessions per week between two consecutive game sessions to a maximal value.
